(19) United States
(12) Patent Application Publication
(10) Pub. No.: US 2022/0001057 A1
(43) Pub. Date: Jan. 6, 2022

(54) ENERGY SUPPLY EQUIPMENT

(71) Applicant: XMIGHT CORPORATION, Taipei City (TW)

(72) Inventors: Yung-Chang Liang, Taipei City (TW); Shu-Hui Tien, Taipei City (TW); Tsung-Tso Kan, Taipei City (TW); Chieh-Ying Pan, Taipei City (TW)

(73) Assignee: XMIGHT CORPORATION, Taipei City (TW)

(21) Appl. No.: 17/036,480

(22) Filed: Sep. 29, 2020

(30) Foreign Application Priority Data
Jul. 3, 2020 (TW) 109122638

(51) Int. Cl. A61L 2/16

(52) U.S. Cl. CPC A61L 2/16; A61L 2202/14

(57) ABSTRACT

An energy supply equipment for supplying energy to an equipment to be powered is provided, including includes a main body, an energy supply unit, and a disinfection device. The main body includes a setting portion; the energy supply unit is put on the setting portion of the main body, and is operable to be detached from the setting portion wherein after detached from the setting portion, the energy supply unit can be connected to and supply energy to an equipment to be powered and the disinfection device is provided on or near the setting portion such that when the energy supply unit is put on the setting portion, the disinfection device is activated to disinfect the energy supply unit on the setting portion.

### BACKGROUND OF THE INVENTION

### 1. Technical Field

The present invention relates generally to an energy supply equipment, and more particularly to an energy supply equipment with disinfection function.

### 2. Description of Related Art

At present, most gas stations are equipped with self-service refueling machines, so that consumers can refuel by themselves rather than waiting when the gas station is understaffed. However, it may cause hygienic problem when different people take the fuel gun for self-refueling.

In view of the harm to social development and the risk for public health, which are caused by infectious diseases, some gas station operators have placed disinfectants next to the refueling machines. Thus, users can disinfect and clean the fuel gun before refueling, thereby improving the sanitary conditions of the fuel gun.

Nevertheless, if users do not perform the disinfection step certainty or forget to perform the disinfection step, it may cause a breach in public health and become a potential path for disease transmission.

Accordingly, an energy supply equipment with disinfection function is urgently needed to improve public health conditions and reduce the chance of disease transmission.

### BRIEF SUMMARY OF THE INVENTION

In view of the above, the primary objective of the present invention is to provide an energy supply equipment with disinfection function, including a sensing component and a disinfection device. When the sensing component detects that an energy supply unit is put on a setting portion, the disinfection device is activated to disinfect the energy supply unit on the setting portion.

The present invention provides an energy supply equipment for supplying energy to an equipment to be powered; the energy supply equipment includes a main body, an energy supply unit, and a disinfection device; the main body includes a setting portion; the energy supply unit is put on the setting portion of the main body and is operable to be detached from the setting portion; after the energy supply unit is detached from the setting portion, the energy supply unit can be connected to and supply energy to an equipment to be powered; the disinfection device is provided on or near the setting portion, wherein when the energy supply unit is put on the setting portion, the disinfection device is activated to disinfect the energy supply unit on the setting portion.

According to a preferred embodiment of the present invention, the main body includes a refueling machine, a charging machine, or a charging station for rechargeable battery pack.

According to a preferred embodiment of the present invention, the energy supply unit includes a fuel gun, a charging gun, or a rechargeable battery pack.

According to a preferred embodiment of the present invention, the energy supply unit has a holding portion to be held by a user's hand so that the energy supply unit can be separated from the setting portion, and furthermore, the energy supply unit can be connected to the equipment to be powered for supply energy to the equipment to be powered.

According to a preferred embodiment of the present invention, the energy supply equipment includes a first sensing component provided on or near the setting portion for sensing whether the energy supply unit is put on the setting portion of the main body, wherein the disinfection device is electrically connected to the first sensing component. When the first sensing component senses that the energy supply unit is put on the setting portion, the disinfection device is activated to disinfect the energy supply unit on the setting portion.

According to a preferred embodiment of the present invention, the first sensing component includes a light sensor, a contact sensor, a magnetic sensor, or an image recognition device. If the first sensing component is the light sensor, the light sensor senses whether the energy supply unit is put on the setting portion of the main body; if the light sensor senses that the energy supply unit is put on the setting portion, the disinfection device is activated to disinfect the energy supply unit on the setting portion. If the first sensing component is the contact sensor and is in contact with the energy supply unit, the contact sensor activates the disinfection device to disinfect the energy supply unit of the setting portion. If the first sensing component is the magnetic sensor and is induced by a magnetic field of a magnetic component on the energy supply unit, the magnetic sensor activates the disinfection device to disinfect the energy supply unit on the setting portion. If the first sensing component is the image recognition device, the image recognition device automatically recognizes whether the energy supply unit is put on the setting portion; if the image recognition device recognizes that the energy supply unit is put on the setting portion, the image recognition device activates the disinfection device to disinfect the energy supply unit on the setting portion.

According to a preferred embodiment of the present invention, if the first sensing component is the image recognition device, the image recognition device is provided for sensing whether a user is close to the main body or would like to take the energy supply unit. When the disinfection device disinfects the energy supply unit, and the image recognition device senses that a user is close to the main body or would like to take the energy supply unit, the disinfection device stops disinfecting the energy supply unit, or alternatively, a display device of the main body instructs the user to take another energy supply unit which has been disinfected.

According to a preferred embodiment of the present invention, when the first sensing component detects that the energy supply unit is put on the setting portion, the disinfection device is activated to disinfect the holding portion of the energy supply unit.

According to a preferred embodiment of the present invention, the main body includes a cover which is controllable to cover the setting portion or be lifted relative to the setting portion. When the cover is lifted relative to the setting portion, the energy supply unit is controllable to be connected to or detached from the setting portion.

According to a preferred embodiment of the present invention, the first sensing component and the disinfection device are set on an inner surface of the cover; the inner surface faces the setting portion.

According to a preferred embodiment of the present invention, the cover includes a partition which divides the inner surface of the cover into a first area and a second area. The first sensing component is set on the first area, and the disinfection device is set on the second area. When the energy supply unit is put on the setting portion, the holding portion of the energy supply unit is covered by the second area of the cover.

According to a preferred embodiment of the present invention, the energy supply equipment includes a timing device which is provided on the main body. The timing device is electrically connected to the disinfection device, and is preset with an automatic disinfection cycle. When the automatic disinfection cycle begins, the timing device activates the disinfection device to disinfect the energy supply unit on the setting portion.

According to a preferred embodiment of the present invention, the timing device is electrically connected to a display device of the main body, wherein the display device shows an automatic disinfection process of the energy supply unit.

According to a preferred embodiment of the present invention, the timing device is electrically connected to a transmission device which uploads and stores the automatic disinfection process of the energy supply unit in a cloud system, wherein the automatic disinfection process is recorded by the timing device. At least one mobile communication device is connected to the transmission device in signal or connected to the cloud system. Users can instantly look up the automatic disinfection process of the energy supply unit through an application software in the at least one mobile communication device.

According to a preferred embodiment of the present invention, the energy supply equipment includes a second sensing component provided on the main body, wherein the second sensing component is provided for sensing whether a user is close to the main body or would like to take the energy supply unit. When the disinfection device disinfects the energy supply unit, and the second sensing component senses that a user is close to the main body or would like to take the energy supply unit, the disinfection device stops disinfecting the energy supply unit, or alternatively, a display device of the main body instructs the user to take another energy supply unit which has been disinfected.

According to a preferred embodiment of the present invention, the disinfection device includes a UV disinfection device, a disinfectant spraying device, or a heating device.

The effect of the present invention is to provide an energy supply equipment with disinfection function, which includes the sensing component and the disinfection device. When the sensing component detects that the energy supply unit is put on the setting portion, the disinfection device is activated to disinfect the energy supply unit on the setting portion.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a perspective view of the energy supply equipment of the first embodiment of the present invention;

FIG. 2 is a lateral view of the energy supply equipment in FIG. 1;

FIG. 3 is a perspective view of the energy supply equipment of the second embodiment of the present invention;

FIG. 4 is a lateral view of the energy supply equipment in FIG. 3;

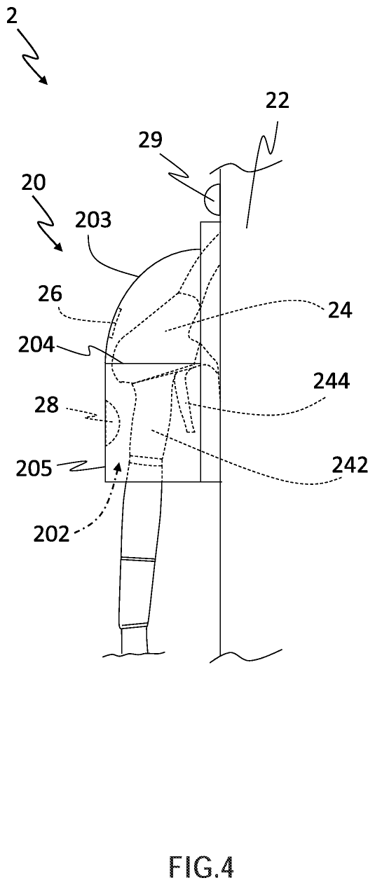

FIG. 5 is a perspective view of the energy supply equipment of the third embodiment of the present invention; and

FIG. 6 is a lateral view of the energy supply equipment in FIG. 5.

### DETAILED DESCRIPTION OF THE INVENTION

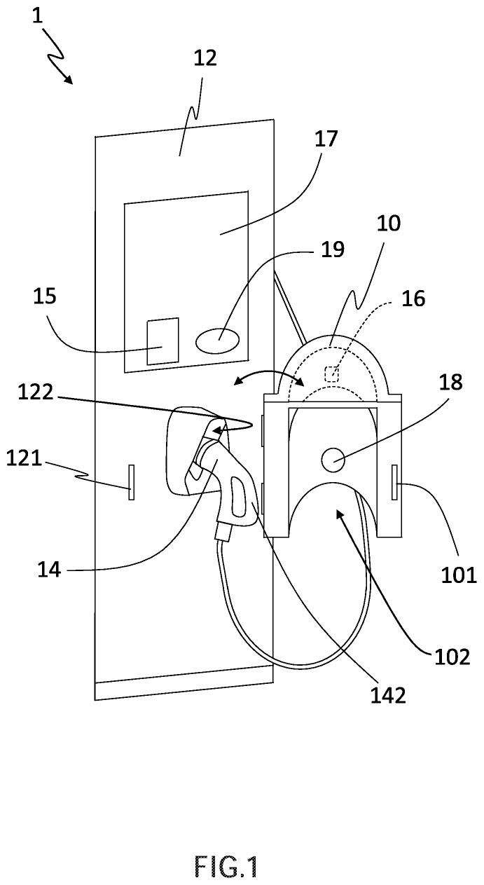

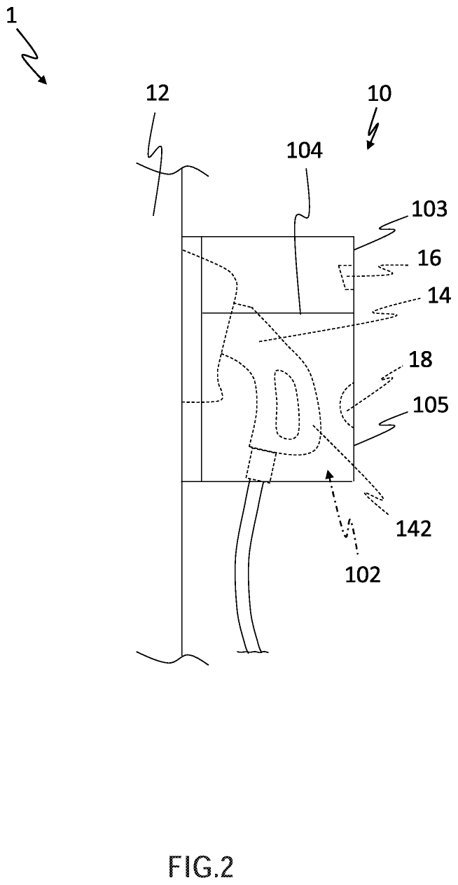

As shown in FIG. 1 and FIG. 2, the first embodiment of the present invention, an energy supply equipment 1, includes a main body 12, an energy supply unit 14, a first sensing component 16, and a disinfection device 18.

The main body 12 includes a setting portion 122. In the first embodiment, the main body 12 can be a charging machine for example, but is not limited to the charging machine.

The energy supply unit 14 is put on the setting portion 122 of the main body 12, and can be detached operably from the setting portion 122. After detached from the setting portion 122, the energy supply unit 14 can be connected to and supply energy to an equipment to be powered (not shown). In the first embodiment, the energy supply unit 14 has a holding portion 142 to be held by a user's hand (not shown) so that the energy supply unit 14 can be separated from the setting portion 122, and furthermore, the energy supply unit 14 can be connected to the equipment to be powered for supply energy to the equipment to be powered. In the first embodiment, the energy supply unit 14 can be a charging gun, and the equipment to be powered can be an electric vehicle, for example instead of limitations. The charging gun is controllable to be connected to a charging port of the electric vehicle so as to supply electrical energy to the power storage device of the electric vehicle.

In the first embodiment, the main body 12 includes a cover 10 which is controllable to cover the setting portion 122 or be lifted relative to the setting portion 122. When the cover 10 is lifted relative to the setting portion 122, the energy supply unit 14 is controllable to be connected to or detached from the setting portion 122. As shown in FIG. 1, the cover 10 can pivot on the main body 12. The main body 12 includes a first locking part 121, while the cover 10 includes a second locking part 101. When the cover 10 covers the setting portion 122, the first locking part 121 and the second locking part 101 are locked to each other, which makes the cover 10 unable to pivot on the main body 12. The cover 10 has an accommodating space 102 for sheltering the setting portion 122 and the energy supply unit 14 put on the setting portion 122.

The first sensing component 16 is provided on or near the setting portion 122 for sensing whether the energy supply unit 14 is put on the setting portion 122 of the main body 12. In the first embodiment, the first sensing component 16 is provided on the inner surface of the cover 10, i.e., the first sensing component 16 is provided near the setting portion 122. When the cover 10 covers the setting portion 122, the inner surface thereof faces the setting portion 122. In the first embodiment, the first sensing component 16 can be a light sensor for example instead of a limitation. If the first sensing component 16 is a light sensor, the light sensor senses whether the energy supply unit 14 is put on the setting portion 122 of the main body 12. If the light sensor senses that the energy supply unit 14 is put on the setting portion 122, the disinfection device 18 is activated to disinfect the energy supply unit 14 on the setting portion 122. On the other hand, if the first sensing component 16 is provided on the setting portion 122, the first sensing component 16 can be a contact sensor. When the contact sensor is in contact with the energy supply unit 14, the contact sensor activates the disinfection device 18 to disinfect the energy supply unit 14 on the setting portion 122.

In other embodiments, when the first sensing component 16 is a magnetic sensor and is induced by the magnetic field of a magnetic component (not shown) on the energy supply unit 14, the magnetic sensor activates the disinfection device 18 to disinfect the energy supply unit 14 on the setting portion 122. In addition, if the first sensing component 16 is an image recognition device, the image recognition device automatically recognizes whether the energy supply unit 14 is put on the setting portion 122; if the image recognition device recognizes that the energy supply unit 14 is put on the setting portion 122, the image recognition device activates the disinfection device 18 to disinfect the energy supply unit 14 on the setting portion 122.

Furthermore, if the first sensing component 16 is an image recognition device, the image recognition device senses whether a user (not shown) is close to the main body 12 or would like to take the energy supply unit 14. When the disinfection device 18 disinfects the energy supply unit 14, and the image recognition device senses that a user is close to the main body 12 or would like to take the energy supply unit 14, the disinfection device 18 stops disinfecting the energy supply unit 14, or alternatively, a display device 17 of the main body 12 instructs the user to take another energy supply unit (not shown) which has been disinfected.

In the first embodiment, when the first sensing component 16 senses that the energy supply unit 14 is put on the setting portion 122, the disinfection device 18 is activated to disinfect the holding portion 142 of the energy supply unit 14.

The disinfection device 18 is provided on or near the setting portion 122, and is electrically connected to the first sensing component 16. In the first embodiment, the disinfection device 18 is provided on the inner surface of the cover 10, i.e., the disinfection device 18 is provided near the setting portion 122. When the first sensing component 16 senses that the energy supply unit 14 is put on the setting portion 122, the disinfection device 18 is activated to disinfect the energy supply unit 14 on the setting portion 122. In the first embodiment, the disinfection device 18 includes a UV disinfection device, a disinfectant spraying device, or a heating device, but is not limited to these devices. In another embodiment, the disinfection device can also be provided on the setting portion so as to disinfect the energy supply unit on the setting portion nearby.

The cover 10 includes a partition 104 which divides the inner surface of the cover 10 into a first area 103 and a second area 105. The first sensing component 16 is provided on the first area 103, and the disinfection device 18 is provided on the second area 105. When the energy supply unit 14 is put on the setting portion 122, the holding portion 142 of the energy supply unit 14 is covered by the second area 105 of the cover 10. Therefore, the disinfection device 18 can be restricted to only perform the disinfection step on the holding portion 142 of the energy supply unit 14, so as to save the setup cost of the disinfection device 18, and to avoid adverse effects on other parts of the energy supply unit 24 which do not need to be disinfected (such as the charger of the charging gun).

Of course, aside from providing the cover, in other embodiments of the present invention, if the main body does not include the cover, the first sensing component and the disinfection device can also be set near the setting portion so as to achieve the same effects of detection and/or disinfection.

In the first embodiment, the energy supply equipment 1 includes a second sensing component 19 provided on the main body 12 for sensing whether a user (not shown) is close to the main body 12 or would like to take the energy supply unit 14. When the disinfection device 18 disinfects the energy supply unit 14, and the second sensing component 19 senses that a user is close to the main body 12 or would like to take the energy supply unit 14, the disinfection device 18 stops disinfecting the energy supply unit 14, or alternatively, the display device 17 of the main body 12 instructs the user to take another energy supply unit (not shown) which has been disinfected.

In the first embodiment, the energy supply equipment 1 includes a timing device 15 provided on the main body 12. The timing device 15 is electrically connected to the disinfection device 18, and is preset with an automatic disinfection cycle. When the automatic disinfection cycle begins, the timing device 15 activates the disinfection device 18 to disinfect the energy supply unit 14 on the setting portion 122. In the first embodiment, the automatic disinfection cycle of the timing device 15 includes activation time point, length of time to disinfect, and interval length of time between two automatic disinfection cycles, but is not limited to include these items.

In the first embodiment, the timing device 15 will automatically record the history (process) of automatic disinfection by the disinfection device 18, so as to record data including the activation time point, length of time to disinfect, and interval length of time between two automatic disinfection cycles in each automatic disinfection. Additionally, the timing device 15 is electrically connected to the display device 17 of the main body 12, and can show the automatic disinfection process of the energy supply unit 11 on the display device 17, so that users can view the history of automatic disinfection by the disinfection device 18. Furthermore, according to the automatic disinfection process by the disinfection device 18, which is recorded by the timing device 15, the maintenance personnel of the energy supply equipment 1 can easily calculate the degree of use of the disinfection device 18, so as to accurately replace the consumables. For example, if the disinfection device 18 is a UV disinfection device, the maintenance personnel can estimate the lifetime of UV lighting instrument according to the automatic disinfection process, and to replace the UV lighting instrument before its life limit. If the disinfection device 18 is a disinfectant spraying device, the maintenance personnel can calculate the amount of the used disinfectant according to the automatic disinfection process, and replenish the disinfectant before it runs out. Thus, the energy supply equipment 1 can be effectively managed and maintained.

In the first embodiment, the timing device 15 can be electrically connected to a transmission device (not shown) which wirelessly uploads and stores the recorded automatic disinfection process of the energy supply unit 11 in a cloud system (not shown). In addition to using wireless transmission, the transmission device can also use wired transmission to achieve the same purpose. Moreover, at least one mobile communication device (not shown) carried by users can be directly connected to the transmission device in signal or connected to the cloud system. Therefore, users can instantly look up the automatic disinfection process of the energy supply unit 11 through an application software (APP) in the at least one mobile communication device.

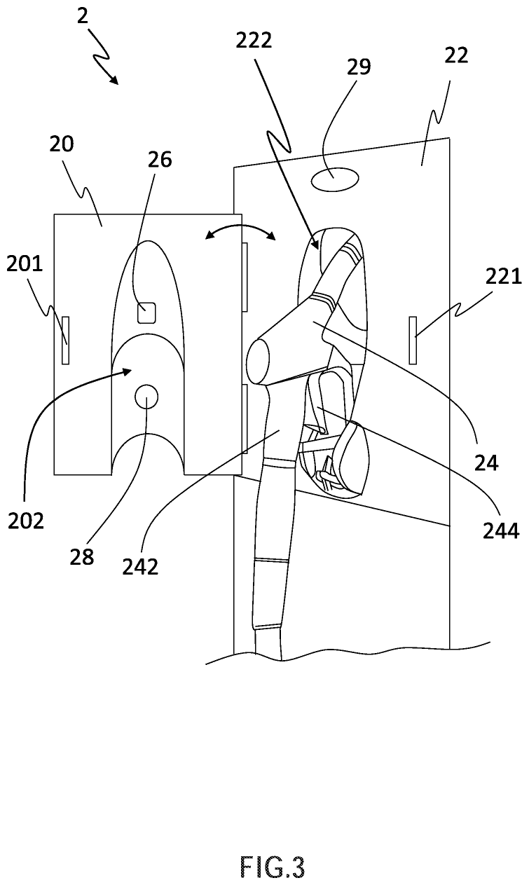

As shown in FIG. 2 and FIG. 3, a second embodiment of the present invention, an energy supply equipment 2 is provided. The energy supply equipment 2 includes a main body 22, an energy supply unit 24, a first sensing component 26, and a disinfection device 28.

The main body 22 has a setting portion 222. In the second embodiment, the main body 22 can be a refueling machine for example, but is not limited to the refueling machine.

The energy supply unit 24 is put on the setting portion 222 of the main body 22, and can be detached operably from the setting portion 222. After detached from the setting portion 222, the energy supply unit 24 can be connected and supply energy to an equipment to be powered (not shown). In this second embodiment, the energy supply unit 24 has a holding portion 242 to be held by a user's hand (not shown) so that the energy supply unit 24 can be separated from the setting portion 222, and furthermore, the energy supply unit 24 can be connected to the equipment to be powered for supply energy to the equipment to be powered. In the second embodiment, the energy supply unit 24 can be a fuel gun, and the equipment to be powered can be a traditional vehicle or motorcycle, for example instead of limitations. The fuel gun is controllable to be connected to a fuel tank port of the traditional vehicle or motorcycle so as to supply gasoline to the traditional vehicle or motorcycle. In the second embodiment, the fuel gun includes an activate key 244. When the fuel gun is connected to the fuel tank port of the traditional vehicle or motorcycle, a user can press the activate key 244 to supply gasoline to the traditional vehicle or motorcycle.

In the second embodiment, the main body 22 includes a cover 20 which is controllable to cover the setting portion 222 or be lifted relative to the setting portion 222. When the cover 20 is lifted relative to the setting portion 222, the energy supply unit 24 is controllable to be connected to or detached from the setting portion 222. As shown in FIG. 3, the cover 20 can pivot on the main body 22. The main body 22 includes a first locking part 221, and the cover 20 includes a second locking part 201. When the cover 20 covers the setting portion 222, the first locking part 221 and the second locking part 201 are locked to each other, which makes the cover 20 unable to pivot on the main body 22. The cover 20 has an accommodating space 202 for sheltering the setting portion 222 and the energy supply unit 24 on the setting portion 222.

The first sensing component 26 is provided on or near the setting portion 222 for sensing whether the energy supply unit 24 is put on the setting portion 222 of the main body 22. In the second embodiment, the first sensing component 26 is provided on the inner surface of the cover 20, i.e., the first sensing component 26 is provided near the setting portion 222. When the cover 20 covers the setting portion 222, the inner surface thereof faces the setting portion 222. In the second embodiment, the first sensing component 26 can be a light sensor for example instead of a limitation. If the first sensing component 26 is a light sensor, the light sensor senses whether the energy supply unit 24 is put on the setting portion 222 of the main body 22. If the light sensor senses that the energy supply unit 24 is put on the setting portion 222, the disinfection device 28 is activated to disinfect the energy supply unit 24 on the setting portion 222. On the other hand, if the first sensing component 26 is provided on the setting portion 222, the first sensing component 26 can be a contact sensor. When the contact sensor is in contact with the energy supply unit 24, the contact sensor activates the disinfection device 28 to disinfect the energy supply unit 24 on the setting portion 222.

In the second embodiment, when the first sensing component 26 senses that the energy supply unit 24 is put on the setting portion 222, the disinfection device 28 is activated to disinfect the holding portion 242 of the energy supply unit 24.

The disinfection device 28 is provided on or near the setting portion 222, and is electrically connected to the first sensing component 26. In the second embodiment, the disinfection device 28 is provided on the inner surface of the cover 20, i.e., the disinfection device 28 is provided near the setting portion 222. When the first sensing component 26 senses that the energy supply unit 24 is put on the setting portion 222, the disinfection device 28 is activated to disinfect the energy supply unit 24 on the setting portion 222. In the second embodiment, the disinfection device 28 includes a UV disinfection device, a disinfectant spraying device, or a heating device, but is not limited to these devices. In another embodiment, the disinfection device can also be provided on the setting portion so as to disinfect the energy supply unit on the setting portion nearby.

In the second embodiment, the cover 20 includes a partition 204 which divides the inner surface of the cover 20 into a first area 203 and a second area 205. The first sensing component 26 is provided on the first area 203, and the disinfection device 28 is provided on the second area 205. When the energy supply unit 24 is put on the setting portion 222, the holding portion 242 and the activate key 244 of the energy supply unit 24 are covered by the second area 205 of the cover 20. Therefore, the disinfection device 28 can be restricted to only perform the disinfection step on the holding portion 242 and the activate key 244 of the energy supply unit 24, so as to save the setup cost of the disinfection device 28, and to avoid adverse effects on other parts of the energy supply unit 24 which do not need to be disinfected (such as the fueling spout of the fuel gun).

Of course, aside from providing the cover, in other embodiments of the present invention, if the main body does not include the cover, the first sensing component and the disinfection device can also be set near the setting portion so as to achieve the same effects of detection and/or disinfection.

In the second embodiment, the energy supply equipment 2 includes a second sensing component 29 provided on the main body 22 for sensing whether a user (not shown) is close to the main body 22 or would like to take the energy supply unit 24. When the disinfection device 28 disinfects the energy supply unit 24, and the second sensing component 29 senses that a user is close to the main body 22 or would like to take the energy supply unit 24, the disinfection device 28 stops disinfecting the energy supply unit 24, or alternatively, the display device (not shown) of the main body 22 instructs the user to take another energy supply unit (not shown) which has been disinfected.

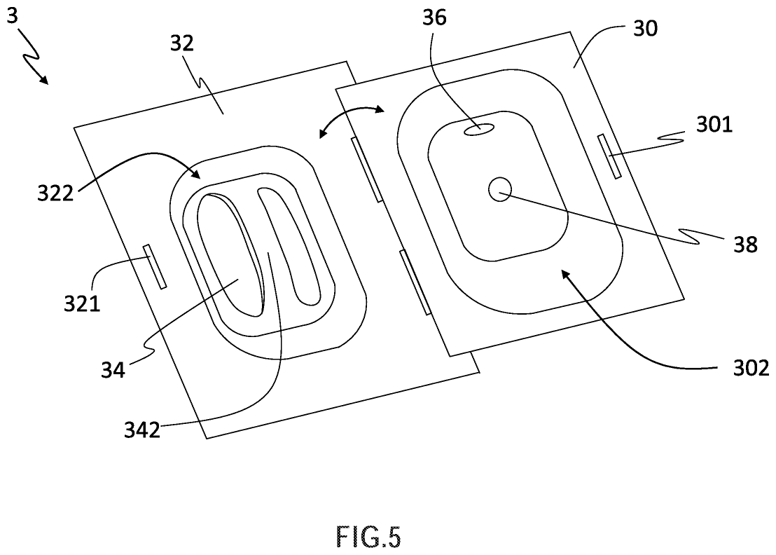

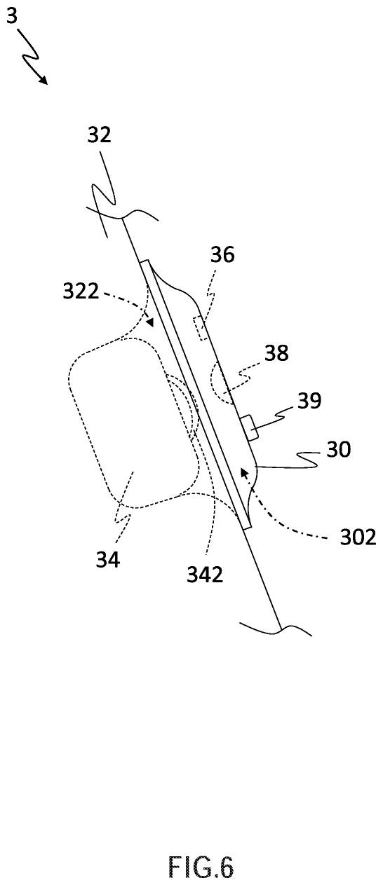

As shown in FIG. 5 and FIG. 6, a third embodiment, an energy supply equipment 3 is provided. The energy supply equipment 3 includes a main body 32, an energy supply unit 34, a first sensing component 36, and a disinfection device 38.

The main body 32 has a setting portion 322. In the third embodiment, the main body 32 can be a charging station for rechargeable battery pack for example, but is not limited to the charging station for rechargeable battery pack.

The energy supply unit 34 is put on the setting portion 322 of the main body 32, and can be detached operably from the setting portion 322. After detached from the setting portion 322, the energy supply unit 34 can be connected and supply energy to an equipment to be powered (not shown). In this third embodiment, the energy supply unit 34 has a holding portion 342 to be held by a user's hand (not shown) so that the energy supply unit 34 can be separated from the setting portion 322, and furthermore, the energy supply unit 34 can be connected to the equipment to be powered for supply energy to the equipment to be powered. In the third embodiment, the energy supply unit 34 can be a rechargeable battery pack, and the equipment to be powered can be an electric scooter, for example instead of limitations. The rechargeable battery pack is controllable to be connected to an accommodating tank for the rechargeable battery pack of the electric scooter so as to supply electrical energy to the electric scooter.

In the third embodiment, the main body 32 includes a cover 30 which is controllable to cover the setting portion 322 or be lifted relative to the setting portion 322. When the cover 30 is lifted relative to the setting portion 322, the energy supply unit 34 is controllable to be connected to or detached from the setting portion 322. As shown in FIG. 5, the cover 30 can pivot on the main body 32. The main body 32 includes a first locking part 321, and the cover 30 includes a second locking part 301. When the cover 30 covers the setting portion 322, the first locking part 321 and the second locking part 301 are locked to each other, which makes the cover 30 unable to pivot on the main body 32. The cover 30 has an accommodating space 302 for sheltering the setting portion 322 and the energy supply unit 34 on the setting portion 322.

In the third embodiment, when the cover 30 covers the setting portion 322, the disinfection device 38 is located above the holding portion 342 of the energy supply unit 34. Thus, the disinfection device 38 can be restricted to only perform the disinfection step on the holding portion 342 of the energy supply unit 34, so as to save the setup cost of the disinfection device 38, and to avoid adverse effects on other parts of the energy supply unit 34 which do not need to be disinfected (such as the box of the rechargeable battery pack).

The first sensing component 36 is set on or near the setting portion 322 for sensing whether the energy supply unit 34 is put on the setting portion 322 of the main body 32. In the third embodiment, the first sensing component 36 is set on the inner surface of the cover 30. i.e., the first sensing component 36 is set near the setting portion 322. When the cover 30 covers the setting portion 322, the inner surface thereof faces the setting portion 322. In the third embodiment, the first sensing component 36 can be a light sensor for example instead of a limitation. If the first sensing component 36 is a light sensor, the light sensor senses whether the energy supply unit 34 is put on the setting portion 322 of the main body 32. If the light sensor senses that the energy supply unit 34 is put on the setting portion 322, the disinfection device 38 is activated to disinfect the energy supply unit 34 on the setting portion 322. On the other hand, if the first sensing component 36 is provided on the setting portion 322, the first sensing component 36 can be a contact sensor. When the contact sensor is in contact with the energy supply unit 34, the contact sensor activates the disinfection device 38 to disinfect the energy supply unit 34 on the setting portion 322.

In this third embodiment, when the first sensing component 36 senses that the energy supply unit 34 is put on the setting portion 322, the disinfection device 38 is activated to disinfect the holding portion 342 of the energy supply unit 34.

The disinfection device 38 is provided on or near the setting portion 322, and is electrically connected to the first sensing component 36. In the third embodiment, the disinfection device 38 is provided on the inner surface of the cover 30, i.e., the disinfection device 38 is provided near the setting portion 322. When the first sensing component 36 senses that the energy supply unit 34 is put on the setting portion 322, the disinfection device 38 is activated to disinfect the energy supply unit 34 on the setting portion 322. In the third embodiment, the disinfection device 38 includes a UV disinfection device, a disinfectant spraying device, or a heating device, but is not limited to these devices. In another embodiment, the disinfection device can also be provided on the setting portion so as to disinfect the energy supply unit on the setting portion nearby.

Of course, aside from providing the cover, in other embodiments of the present invention, if the main body does not include the cover, the first sensing component and the disinfection device can also be set near the setting portion so as to achieve the same effects of detection and/or disinfection.

In the third embodiment, the energy supply equipment 3 includes a second sensing component 39 provided on the main body 32 for sensing whether a user (not shown) is close to the main body 32 or would like to take the energy supply unit 34. When the disinfection device 38 disinfects the energy supply unit 34, and the second sensing component 39 senses that a user is close to the main body 32 or would like to take the energy supply unit 34, the disinfection device 38 stops disinfecting the energy supply unit 34, or alternatively, the display device (not shown) of the main body 32 instructs the user to take another energy supply unit (not shown) which has been disinfected.

With the abovementioned design of the present invention, the energy supply equipment with disinfection function includes the sensing component and the disinfection device; when the sensing component detects that the energy supply unit is put on the setting portion, the disinfection device is activated to disinfect the energy supply unit on the setting portion.

It must be noted that the embodiments described above are only preferred embodiments of the present invention. All equivalent structures which employ the concepts disclosed in this specification and the appended claims should fall within the scope of the present invention.

What is claimed is:

1. An energy supply equipment for supplying energy to an equipment to be powered, comprising:
a main body comprising a setting portion;
an energy supply unit which is put on the setting portion of the main body and is operable to be detached from the setting portion, wherein after the energy supply unit is detached from the setting portion, the energy supply unit can be connected to and supply energy to an equipment to be powered; and
a disinfection device provided on or near the setting portion, wherein when the energy supply unit is put on the setting portion, the disinfection device is activated to disinfect the energy supply unit on the setting portion.

2. The energy supply equipment of claim 1, wherein the main body comprises a refueling machine, a charging machine, or a charging station for rechargeable battery pack.

3. The energy supply equipment of claim 1, wherein the energy supply unit comprises a fuel gun, a charging gun, or a rechargeable battery pack.

4. The energy supply equipment of claim 1, wherein the energy supply unit has a holding portion to be held by a user's hand so that the energy supply unit can be separated from the setting portion, and furthermore, the energy supply unit can be connected to the equipment to be powered for supply energy to the equipment to be powered.

5. The energy supply equipment of claim 4, further comprising a first sensing component provided on or near the setting portion for sensing whether the energy supply unit is put on the setting portion of the main body, wherein the disinfection device is electrically connected to the first sensing component; when the first sensing component senses that the energy supply unit is put on the setting portion, the disinfection device is activated to disinfect the energy supply unit on the setting portion.

6. The energy supply equipment of claim 5, wherein the first sensing component comprises a light sensor, a contact sensor, a magnetic sensor, or an image recognition device; if the first sensing component is the light sensor, the light sensor senses whether the energy supply unit is put on the setting portion of the main body; if the light sensor senses that the energy supply unit is put on the setting portion, the disinfection device is activated to disinfect the energy supply unit on the setting portion; if the first sensing component is the contact sensor and is in contact with the energy supply unit, the contact sensor activates the disinfection device to disinfect the energy supply unit of the setting portion; if the first sensing component is the magnetic sensor and is induced by a magnetic field of a magnetic component on the energy supply unit, the magnetic sensor activates the disinfection device to disinfect the energy supply unit on the setting portion; if the first sensing component is the image recognition device, the image recognition device automatically recognizes whether the energy supply unit is put on the setting portion; if the image recognition device recognizes that the energy supply unit is put on the setting portion, the image recognition device activates the disinfection device to disinfect the energy supply unit on the setting portion.

7. The energy supply equipment of claim 6, wherein if the first sensing component is the image recognition device, the image recognition device is provided for sensing whether a user is close to the main body or would like to take the energy supply unit; when the disinfection device disinfects the energy supply unit, and the image recognition device senses that a user is close to the main body or would like to take the energy supply unit, the disinfection device stops disinfecting the energy supply unit, or alternatively, a display device of the main body instructs the user to take another energy supply unit which has been disinfected.

8. The energy supply equipment of claim 5, wherein when the first sensing component detects that the energy supply unit is put on the setting portion, the disinfection device is activated to disinfect the holding portion of the energy supply unit.

9. The energy supply equipment of claim 5, wherein the main body comprises a cover which is controllable to cover the setting portion or be lifted relative to the setting portion; when the cover is lifted relative to the setting portion, the energy supply unit is controllable to be connected to or detached from the setting portion.

10. The energy supply equipment of claim 9, wherein the first sensing component and the disinfection device are set on an inner surface of the cover; the inner surface faces the setting portion.

11. The energy supply equipment of claim 10, wherein the cover comprises a partition which divides the inner surface of the cover into a first area and a second area; the first sensing component is set on the first area, and the disinfection device is set on the second area; when the energy supply unit is put on the setting portion, the holding portion of the energy supply unit is covered by the second area of the cover.

12. The energy supply equipment of claim 4, further comprising a timing device which is provided on the main body, wherein the timing device is electrically connected to the disinfection device; the timing device is preset with an automatic disinfection cycle; when the automatic disinfection cycle begins, the timing device activates the disinfection device to disinfect the energy supply unit on the setting portion.

13. The energy supply equipment of claim 12, wherein the timing device is electrically connected to a display device of the main body; the display device shows an automatic disinfection process of the energy supply unit.

14. The energy supply equipment of claim 13, wherein the timing device is electrically connected to a transmission device which uploads and stores the automatic disinfection process of the energy supply unit in a cloud system, wherein the automatic disinfection process is recorded by the timing device; at least one mobile communication device is connected to the transmission device in signal or connected to the cloud system; users can instantly look up the automatic disinfection process of the energy supply unit through an application software in the at least one mobile communication device.

15. The energy supply equipment of claim 1, further comprising a second sensing component provided on the main body, wherein the second sensing component is provided for sensing whether a user is close to the main body or would like to take the energy supply unit; when the disinfection device disinfects the energy supply unit, and the second sensing component senses that a user is close to the main body or would like to take the energy supply unit, the disinfection device stops disinfecting the energy supply unit, or alternatively, a display device of the main body instructs the user to take another energy supply unit which has been disinfected.

16. The energy supply equipment of claim 1, wherein the disinfection device comprises a UV disinfection device, a disinfectant spraying device, or a heating device.
